(19) United States
(12) Patent Application Publication
(10) Pub. No.: US 2026/0016430 A1
(43) Pub. Date: Jan. 15, 2026

(54) GAS SENSOR CHIP FOR THERMAL CONDUCTIVITY MEASUREMENTS AND METHODS FOR PRODUCING AND OPERATING SUCH

(71) Applicant: Infineon Technologies AG, Neubiberg (DE)

(72) Inventors: Sophie EMPERHOFF, Regensburg (DE); Matthias EBERL, Taufkirchen (DE); Julia Isabel PEREZ BARRAZA, Unterhaching (DE)

(21) Appl. No.: 19/256,707

(22) Filed: Jul. 1, 2025

(30) Foreign Application Priority Data
Jul. 15, 2024 (DE) 102024120164.9

(51) Int. Cl. G01N 25/18; G01N 33/00

(52) U.S. Cl. CPC G01N 25/18; G01N 33/0016; G01N 33/0031; G01N 33/0073

(57) ABSTRACT

A gas sensor chip for carrying out a thermal conductivity measurement includes: a measuring cavity which has an opening so that an ambient gas can flow into the measuring cavity, a reference cavity which is filled with a reference gas and hermetically sealed, a first and a second measuring cavity bar, which are arranged next to one another and free-standing in the measuring cavity, a first and a second reference cavity bar, which are arranged next to one another and free-standing in the reference cavity, wherein each of the measuring cavity bars and each of the reference cavity bars has a first and a second conductor element, which are electrically insulated from each other and which can, based on an operating mode of the gas sensor chip, in each case be operated as a sensor element and/or as a heating element for a thermal conductivity measurement on the ambient gas.

### CROSS-REFERENCE TO RELATED APPLICATION

This application claims priority to Germany Patent Application No. 102024120164.9 filed on Jul. 15, 2024, the content of which is incorporated by reference herein in its entirety.

### TECHNICAL FIELD

The present disclosure relates to a gas sensor chip for carrying out thermal conductivity measurements and methods for producing such a gas sensor chip and methods for operating such a gas sensor chip.

### BACKGROUND

Gas sensor chips for thermal conductivity measurements can be used in a wide range of fields of application, e.g., in the automotive sector, in the industrial sector or else in the consumer sector. Furthermore, such gas sensor chips may be configured to detect and quantify a wide range of gases, such as hydrogen, carbon dioxide, sulfur dioxide, refrigerant gases, etc. To detect such an ambient gas, this gas is passed into a measuring cavity of the gas sensor chip, a heating element is heated and the change of the thermal conductivity in the measuring cavity is compared with the thermal conductivity in a hermetically sealed reference cavity. The sensitivity of the gas sensor chip can in this case be increased by a higher temperature at the sensor element. However, it would be desirable that such gas sensor chips heat up even more strongly and/or offer additional diagnostic options, e.g., for detecting faulty components or for providing further measurement parameters, and/or the option of drift correction. Improved gas sensor chips, improved methods for producing gas sensor chips, and improved methods for operating such gas sensor chips can help solve these and further problems.

### SUMMARY

Various aspects relate to a gas sensor chip for carrying out a thermal conductivity measurement, having: a measuring cavity which has an opening so that an ambient gas can flow into the measuring cavity, a reference cavity which is filled with a reference gas and hermetically sealed, a first and a second measuring cavity bar, which are arranged next to one another and free-standing in the measuring cavity, a first and a second reference cavity bar, which are arranged next to one another and free-standing in the reference cavity, wherein each of the measuring cavity bars and the reference cavity bars in each case has a first and a second conductor element, which are electrically insulated from each other and which can, based on an operating mode of the gas sensor chip, in each case be operated as a sensor element and/or as a heating element for a thermal conductivity measurement on the ambient gas.

Various aspects relate to a method for producing a gas sensor chip for thermal conductivity measurements, wherein the method includes: providing a wafer, forming a measuring cavity in the wafer, which has an opening so that an ambient gas can flow into the measuring cavity, forming a reference cavity in the wafer, filling the reference cavity with a reference gas and hermetically sealing the reference cavity, forming a first and a second measuring cavity bar in the measuring cavity such that they are next to one another and free-standing, forming a first and a second reference cavity bar in the reference cavity such that they are next to one another and free-standing, forming a first and a second conductor element in each case on or in each of the measuring cavity bars and the reference cavity bars in such a way that the first and second conductor elements are electrically insulated from each other in each case and can, based on an operating mode of the gas sensor chip, in each case be operated as a sensor element and/or as a heating element for a thermal conductivity measurement on the ambient gas.

Various aspects relate to a method for operating a gas sensor chip for thermal conductivity measurements, wherein the method includes: providing a gas sensor chip having: a measuring cavity which has an opening so that an ambient gas can flow into the measuring cavity, a reference cavity which is filled with a reference gas and hermetically sealed, a first and a second measuring cavity bar, which are arranged next to one another and free-standing in the measuring cavity, a first and a second reference cavity bar, which are arranged next to one another and free-standing in the reference cavity, wherein each of the measuring cavity bars and the reference cavity bars in each case has a first and a second conductor element, which are electrically insulated from each other and which can, based on an operating mode of the gas sensor chip, in each case be operated as a sensor element and/or as a heating element for a thermal conductivity measurement on the ambient gas; heating up the measuring cavity and the reference cavity using the heating elements; and carrying out the thermal conductivity measurement using the sensor elements.

A person skilled in the art will discern further features and advantages of the implementation upon reading the following detailed description and examining the attached drawings.

### BRIEF DESCRIPTION OF THE DRAWINGS

The present disclosure is shown in an example and non-limiting manner in the illustrations of the attached drawings, in which identical reference numbers refer to similar or identical elements. The elements in the drawings are not necessarily depicted to scale in relation to each other. The features of the various examples shown can be combined, provided that they are not mutually exclusive.

FIG. 1 shows a schematic cross-sectional view of a gas sensor chip having a measuring cavity having two measuring cavity bars and a reference cavity having two reference cavity bars.

FIG. 2 shows a cross section through the measuring cavity and the reference cavity in plan view. As shown in FIG. 2, each of the measuring cavity bars and the reference cavity bars has a first and a second conductor element, which can be used for heating or for measuring.

FIG. 3 shows a schematic cross section through a measuring cavity bar according to one example, in which the two conductor elements comprise different materials or material compositions.

FIGS. 4A and 4B show various electrical circuit arrangements which can be provided with the first and second conductor elements, namely Wheatstone bridge circuit arrangements (FIG. 4A) and parallel circuit arrangements (FIG. 4B).

FIG. 5 shows various operating modes in which the gas sensor chip can be operated in table form, wherein in the various operating modes, two circuits, of which the first and second conductor elements are part, each have different combinations of circuit states.

FIG. 6 shows a schematic cross section through a measuring cavity bar according to a further example, in which the first and second conductor elements are formed next to one another in a semiconductor material of the measuring cavity bar.

FIG. 7 shows a schematic cross section through a measuring cavity bar according to a further example, in which the first and second conductor elements are formed next to one another in a semiconductor material of the measuring cavity bar and an additional heating element is formed in a metallization over the conductor elements.

FIG. 8 shows a schematic cross section through a measuring cavity bar according to a further example, in which the first and second conductor elements are formed next to one another in a metallization on the measuring cavity bar and an additional heating element is formed in a semiconductor material of the measuring cavity bar.

FIGS. 9A to 9F show various options for how, by adjusting an electrical circuit arrangement, a position of the first and second conductor elements in the first and second circuit can be exchanged to provide additional diagnostic options.

FIG. 10 shows a gas sensor which has the gas sensor chip from FIG. 1 and FIG. 2.

FIG. 11 is a flowchart of an example method for producing a gas sensor chip.

FIG. 12 is a flowchart of an example method for operating a gas sensor chip.

### DETAILED DESCRIPTION

The following detailed description refers to the drawings and the examples shown in same. However, it is apparent to a person skilled in the art that one or more aspects of the disclosure can be realized with a lesser degree of specific detail. In other cases, known structures and elements are shown in schematic form to facilitate a description of one or more aspects of the disclosure.

Insofar as the terms “contain”, “have”, “with” or other variations thereof are used either in the detailed description or in the claims, these terms should furthermore have an inclusive meaning in a similar way to the term “comprise”. The terms “coupled” and “connected” along with their derivatives can be used. These terms can be used to indicate that two elements cooperate or interact with each other, wherein it is unimportant whether they are in direct physical or electrical contact with each other or are not in direct contact with each other; intermediate elements or layers can be provided between the “bonded”, “attached” or “connected” elements. Furthermore, the term “example” should mean an example and not the best or optimum.

An efficient gas sensor chip, an efficient method for producing gas sensor chips, and an efficient method for operating gas sensor chips can e.g., reduce material consumption, chemical wastes or ohmic losses and thus enable energy and/or resource savings. Improved gas sensor chips, improved methods for producing gas sensor chips and improved methods for operating gas sensor chips, as specified in this description, can thus contribute at least indirectly to green technology solutions, e.g., to climate-friendly solutions that enable a reduction in energy and/or resource consumption.

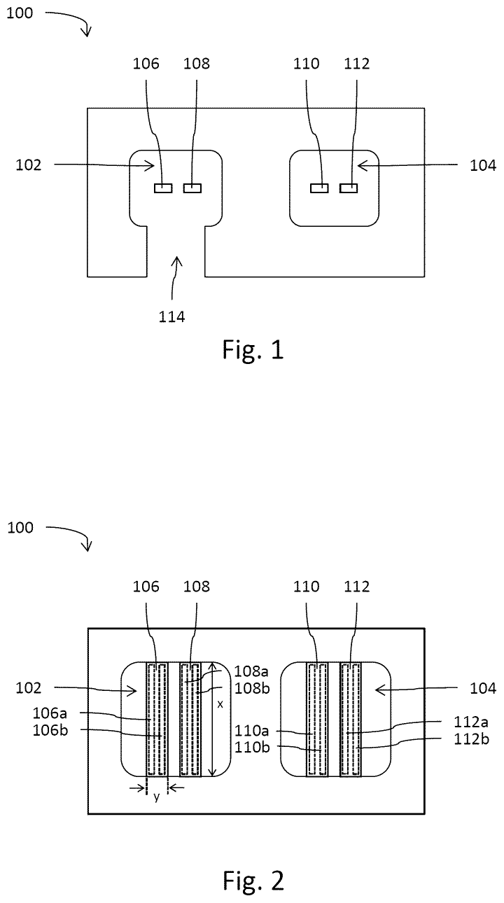

FIG. 1 shows a schematic cross-sectional view of a gas sensor chip 100 for carrying out a thermal conductivity measurement. The gas sensor chip 100 has a measuring cavity 102 and a reference cavity 104. The measuring cavity 102 has an opening 114 so that an ambient gas can flow into the measuring cavity 102. The reference cavity 104 by contrast is filled with a reference gas and hermetically sealed, e.g., no ambient gas can pass into the reference cavity 104.

The known measuring principle of the gas sensor chip 100 is based on the detection of resistance changes of electrically heated resistors. The resistors are thermally decoupled from the rest of the substrate of the gas sensor chip 100 so that the thermal energy is largely discharged into the ambient gas or reference gas surrounding the resistors. Different gases have different coefficients of thermal conductivity and can thus be detected by the gas sensor chip 100.

The gas sensor chip 100 can be configured for use in a suitable gas sensor. The gas sensor chip 100 can be configured e.g., to detect and/or quantify a gas such as hydrogen, carbon dioxide, sulfur dioxide, inert gases, R32, R1234yf, R454, R744, etc. The gas sensor chip 100 can be configured e.g., for use in the automotive sector, in the industrial sector, in the consumer sector, etc. The gas sensor 100 can be configured e.g., to detect a hydrogen leak in an automobile. According to one example, the gas sensor chip 100 has a silicon chip (e.g., the measuring cavity 102 and the reference cavity 104 are formed at least partially in silicon).

As shown in FIG. 1, the measuring cavity 102 has a first measuring cavity bar 106 and a second measuring cavity bar 108. The first and second measuring cavity bars 106, 108 can be arranged in a freely suspended manner, e.g., centrally in the measuring cavity 102 (this may mean that the opposite ends of the measuring cavity bars 106, 108 are connected to a wall of the measuring cavity 102 and span a width of the measuring cavity 102 in a freely suspended manner.

In a manner comparable to the measuring cavity 102, the reference cavity 104 has a first reference cavity bar 110 and a second reference cavity bar 112. These can likewise be arranged in a freely suspended manner and e.g., centrally in the reference cavity 104. In particular, it is possible that the structure and/or the dimensions of the measuring cavity 102 and the reference cavity 104 or the measuring cavity bars 106, 108 and the reference cavity bars 110, 112 are identical, except for the presence of the opening 114 in the measuring cavity 102.

FIG. 2 shows a plan view of the measuring cavity 102 and the reference cavity 104 from above. As shown in FIG. 2, the first and the second measuring cavity bars 106, 108 or the first and the second reference cavity bars 108, 110 can be arranged e.g., parallel to each other. The measuring cavity bars 106, 108 and reference cavity bars 110, 112 can have any suitable shape or any suitable dimensioning. For example, the measuring cavity bars 106, 108 and reference cavity bars 110, 112 can have a length x in a range of approx. 300 μm to approx. 1.5 mm, e.g., approximately 500 μm, approximately 800 μm or approximately 1 mm. The measuring cavity bars 106, 108 and reference cavity bars 110, 112 can have e.g., a width y in a range of approx. 10 μm to approx. 100 μm, e.g., approximately 30 μm, approximately 50 μm or approximately 70 μm. Furthermore, the measuring cavity bars 106, 108 and reference cavity bars 110, 112 can have e.g., a thickness in a range of approx. 1 μm to approx. 10 μm, e.g., approximately 3 μm, approximately 5 μm or approximately 7 μm (wherein the thickness is measured perpendicular to the length x and width y).

According to one example, the measuring cavity 102 and the reference cavity 104 are formed partially or completely in a suitable semiconductor material such as e.g., silicon. In this case, the measuring cavity bars 106, 108 and the reference cavity bars 110, 112 can likewise consist of this semiconductor material (wherein it is possible that e.g., a suitable metallization is arranged on the semiconductor material of the measuring and reference cavity bars 106-112. In particular, in this case, the measuring and reference cavity bars 106-112 may be formed monolithically with the semiconductor material of the side walls of the measuring cavity 102 or the reference cavity 104.

Each of the measuring cavity bars 106, 108 and the reference cavity bars 110, 112 in each case has a first conductor element 106a, 108a, 110a, 112a and a second conductor element 106b, 108b, 110b, 112b. In each of the measuring cavity bars 106, 108 and the reference cavity bars 110, 112, the first conductor elements 106a, 108a, 110a, 112a are in each case electrically insulated from the second conductor elements 106b, 108b, 110b, 112b. In other words, the first conductor elements 106a, 108a, 110a, 112a can be e.g., part of a first circuit and the second conductor elements 106b, 108b, 110b, 112b can be e.g., part of a second circuit, wherein the two circuits are separated from each other.

It should be noted that in the schematic illustration of FIG. 2, the first and second conductor elements of the measuring cavity bars 106, 108 and the reference cavity bars 110, 112 are shown as arranged next to one another on the respective bar, as viewed from above. However, as shown below, it is also possible that the first and second conductor elements are arranged one below the other on the respective bar.

According to one example, the first conductor elements 106a, 108a, 110a, 112a can consist of a first material or a first material composition and the second conductor elements 106b, 108b, 110b, 112b can consist of a second material or a second material composition which is different from the first material or the first material composition. For example, the first conductor elements 106a, 108a, 110a, 112a can consist of a doped semiconductor material (e.g., Si) or comprise one such doped semiconductor material and the second conductor elements 106b, 108b, 110b, 112b can consist e.g., of a metal (e.g., Ag, Al, Au or Cu) or a metal alloy or comprise one such metal or one such metal alloy. According to another example, the first and second conductor elements consist of the same material or the same material composition.

The first conductor elements 106a, 108a, 110a, 112a and the second conductor elements 106b, 108b, 110b, 112b can, based on an operating mode of the gas sensor chip 100, in each case be operated as a sensor element or as a heating element for a thermal conductivity measurement on the ambient gas. Operation as sensor elements can mean that the first conductor elements 106a, 108a, 110a, 112a of the first circuit or the second conductor elements 106b, 108b, 110b, 112b of the second circuit are in each case connected in a known manner to form a Wheatstone bridge circuit arrangement in order to carry out a thermal conductivity measurement on the ambient gas. Operation as heating elements can in turn mean that the first conductor elements 106a, 108a, 110a, 112a of the first circuit or the second conductor elements 106b, 108b, 110b, 112b of the second circuit are in each case parallel-connected in order to heat the ambient gas or the reference gas. It is further possible that the first circuit or the second circuit is in an open state, e.g., no voltage is applied at the respective conductor elements.

As explained in more detail below, based on an operating mode of the gas sensor chip 100, the aforementioned states of the two circuits can be combined in different ways (e.g., in one operating mode, the first circuit can provide a Wheatstone bridge circuit arrangement and the second circuit can provide a heating circuit arrangement or the first circuit can be in the open state and the second circuit can provide a Wheatstone bridge circuit arrangement, etc.). The capability of the gas sensor chip 100 to combine different states of the two circuits in various ways can allow e.g., additional diagnostic options, improved sensitivity, drift compensation, etc., as is also explained in more detail below.

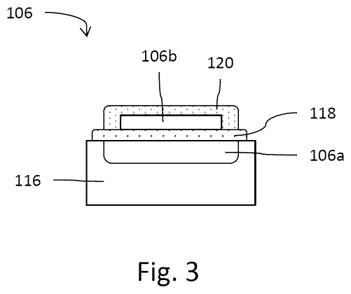

FIG. 3 shows a detailed illustration of the cross section of the first measuring cavity bar 106 according to a specific example. The second measuring cavity bar 108 or the reference cavity bars 110, 112 can have an identical structure. In particular, in the example of FIG. 3, the first conductor element 106a and the second conductor element 106b are arranged one below the other.

In the example of FIG. 3, the first conductor element 106a is formed by a doped semiconductor material which is formed in a semiconductor substrate 116 of the first measuring cavity bar 106. The second conductor element 106b is arranged above the first conductor element 106a. The second conductor element 106b is formed by a metal or a metal alloy which is deposited on the semiconductor substrate 116. The conductor elements 106a, 106b are electrically insulated from each other by an intermediate layer 118. The intermediate layer 118 can be any suitable electrically insulating material. For example, the intermediate layer 118 may comprise a nitride or consist of the same, e.g., silicon nitride, or the intermediate layer 118 may comprise an oxide or consist of the same, e.g., silicon oxide. The intermediate layer 118 can have any suitable thickness, e.g., a thickness in the nanometer range or a thickness in the micrometer range.

According to the example shown in FIG. 3, the second conductor element 106b can be covered by a cover layer 120. The cover layer 120 can be formed e.g., as a protective layer which protects the second conductor element 106b (and possibly also the first conductor element 106a) from environmental influences (e.g., from the ambient gas). The intermediate layer 118 can be formed in a comparable manner as a protective layer. The cover layer 120 can be a passivation layer. The cover layer 120 can comprise e.g., a nitride or an oxide or consist of the same, e.g., silicon nitride or silicon oxide. The cover layer 120 may have comparable dimensions to the intermediate layer 118.

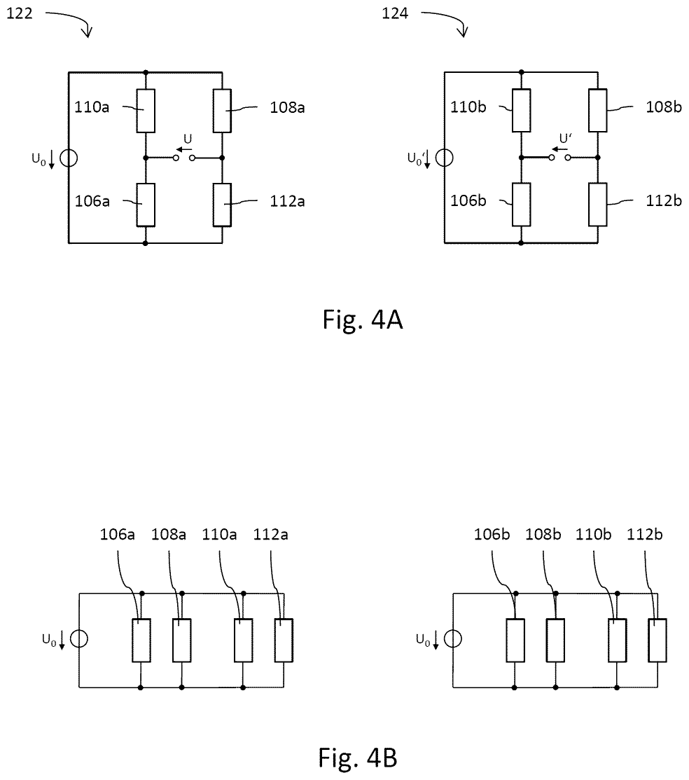

FIGS. 4A and 4B show example electrical circuit arrangements which the first conductor elements 106a, 108a, 110a, 112a and the second conductor elements 106b, 108b, 110b, 112b can be connected to form, based on an operating mode of the gas sensor chip 100. The first conductor elements 106a, 108a, 110a, 112a here form a first circuit 122 and the second conductor elements 106b, 108b, 110b, 112b form an independent second circuit 124.

FIG. 4A by way of example shows for both circuits 122, 124 how the respective conductor elements can be connected to form a Wheatstone bridge circuit arrangement. In this state, the respective circuit of the first and second circuits 122, 124 can be operated for thermal conductivity measurement on the ambient gas. FIG. 4B by way of example shows for both circuits 122, 124 how the respective conductor elements can be parallel-connected. In this state, the respective circuit of the first and second circuits 122, 124 can be operated as a heating circuit for the measuring cavity 102 or the reference cavity 104.

Depending on an operating mode of the gas sensor chip 100, the two circuits 122, 124 can be operated in different combinations; in the Wheatstone bridge circuit arrangement according to FIG. 4A, in the parallel circuit arrangement according to FIG. 4B, and in an open state (e.g., no voltage is applied at the corresponding circuit).

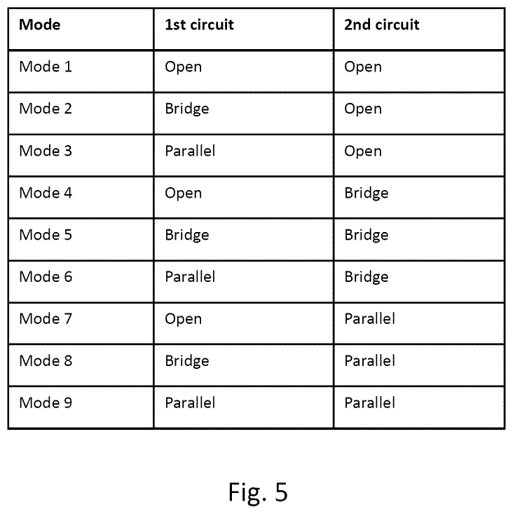

In FIG. 5, nine different example operating modes of the gas sensor chip 100 are listed in table form. Here, “open” means that no voltage is applied at the respective circuit, “bridge” means that the respective circuit is in the Wheatstone bridge circuit arrangement (e.g., can be used as a measuring circuit) and “parallel” means that the respective circuit is in the parallel circuit arrangement (e.g., can be used as a heating circuit). The numbering of modes 1 through 9 is purely arbitrary and should not imply a particular ranking.

In mode 1, both circuits 122, 124 are in the switched-off state, e.g., the gas sensor chip 100 itself is switched off. In mode 5, both circuits are operated in the Wheatstone bridge circuit arrangement, e.g., two mutually independent measurements can be performed. In mode 9, both circuits 122, 124 are in the parallel-connected state, e.g., with this mode the maximum heating power can be achieved. One of modes 2, 3, 4 and 7 can be used e.g., if only one of circuits 122, 124 is required for measuring or for heating and the other of the circuits 122, 124 can remain switched off. One of the modes 6 and 8 can be used if a measurement is to be performed and the ambient gas and the reference gas should be heated at the same time.

Since the gas sensor chip 100 has the two circuits 122, 124 with the possible states shown in FIG. 5, additional options for measurement or diagnostics arise compared to a conventional device. For example, it is possible with the gas sensor chip 100 to carry out measurements at different temperatures, for example by using both circuits 122, 124 or only one of the circuits 122, 124 for heating. In the process, use can be made of the thermal inertia of the gas sensor chip 100, in order, e.g., after heating using both circuits 122, 124 in mode 9, to switch to another mode in which at least one of the circuits 122, 124 is operated as a Wheatstone bridge (that is to say e.g., mode 8 or mode 5). Due to the thermal inertia, the temperature drops only gradually and not abruptly after such a switch. By measuring at a higher temperature, it is possible to increase e.g., the sensitivity of the gas sensor chip 100.

An example measurement cycle, e.g., a sequence of operating modes for carrying out a measurement, could proceed as follows: mode 1→mode 3→mode 2→mode 1, wherein in this sequence the measurement takes place in mode 2 and heating takes place in mode 3, which takes place before that. An example measurement cycle for measuring at a higher temperature can proceed as follows: mode 1→mode 9→mode 5→mode 1, wherein the measurement takes place in mode 5.

Example measurement cycles at comparatively low temperatures are the following: mode 1→mode 2 (measurement)→mode 1; and mode 1→mode 3→mode 2 (measurement)→mode 1. Example measurement cycles at comparatively moderate temperatures are: mode 1→mode 5 (measurement)→mode 1; and mode 1→mode 8 (measurement)→mode 1. Example measurement cycles at comparatively high temperatures are: mode 1→mode 6→mode 2 (measurement)→mode 1; and mode 1→mode 9→mode 2 (measurement)→mode 1.

As mentioned above, the two circuits 122, 124 can be used to carry out two independent measurements, wherein the measuring elements 106a and 106b or 108a and 108b or 110a and 110b or 112a and 112b are thermally coupled however. This can be used e.g., to compensate a drift in the gas sensor chip 100. Example measurement cycles for such a redundant measurement are: mode 1→mode 5 (measurement)→mode 1; and mode 1→mode 9→mode 5 (measurement)→mode 1.

As mentioned above, the gas sensor chip 100 can be used to carry out measurements at different temperatures, e.g., initially at a comparatively low temperature and subsequently at a comparatively higher temperature. The first measurement at low temperature can be used e.g., to ascertain an offset of the gas sensor chip 100 with minimal influence by the ambient gas or the reference gas. The second measurement at higher temperature can then be used to measure the gas effects. Furthermore, due to the option of setting different temperatures in the measuring cavity 102 or the reference cavity 104, a temperature spectroscopy can be carried out on the ambient gas.

Example measurement cycles for such measurements at different temperatures are: mode 1→mode 2 (first measurement)→mode 3→mode 2 (second measurement)→mode 1; and mode 1→mode 5 (first measurement)→mode 9→mode 5 (second measurement)→mode 1. Here, the first measurement in each case takes place at a low temperature and the second measurement takes place at a higher temperature.

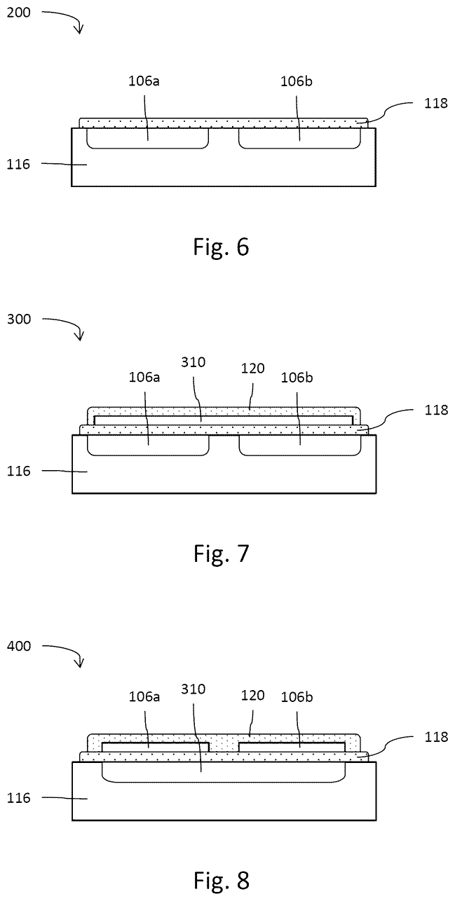

FIG. 6 shows a schematic cross section through a measuring cavity bar 200 which may be similar or identical to the measuring cavity bar 106 except for the differences described below. The measuring cavity bar 200 can be present in place of the first measuring cavity bar 106 in the gas sensor chip 100. Similarly, the second measuring cavity bar 108 and the reference cavity bars 110, 112 can also be replaced by structures identical to the measuring cavity bar 200.

In contrast to the measuring cavity bar 106 (cf. FIG. 3), in the measuring cavity bar 200, the two conductor elements 106a, 106b can be arranged next to one another instead of one below the other. In particular however, the two conductor elements 106a, 106b of the measuring cavity bar 200 can consist of the same material or the same material composition, e.g., of a suitably doped semiconductor material. Since the two conductor elements 106a, 106b are produced by the same process, it is possible to ensure that the two conductor elements 106a, 106b can have identical or almost identical electrical resistance values. In this case, the first conductor element 106a and the second conductor element 106b are interchangeable between the two circuits 122, 124 without distorting the measurement result.

FIG. 7 shows a schematic cross section through a further measuring cavity bar 300 which is similar or identical to the measuring cavity bar 200 except for the differences described below.

In particular, in addition to the two conductor elements 106a, 106b, the measuring cavity bar 300 also has an additional heating element 310 which is arranged on the measuring cavity bar 300. The heating element 310 can be electrically insulated from the conductor elements 106a, 106b e.g., by the intermediate layer 118. As shown in FIG. 7, the additional heating element 310 can be arranged above the conductor elements 106a, 106b and can consist of another material or another material composition, in particular a metal or a metal alloy.

Thanks to the additional heating element 310, both conductor elements 106a, 106b can be used simultaneously for measuring while the heating element 310 is used for heating the ambient gas or the reference gas. Alternatively, one or both of the conductor elements 106a, 106b can be used in addition to the heating element 310 for heating, as a result of which it is possible to increase the temperature that can be achieved. This can improve e.g., the sensitivity of the gas sensor chip 100.

FIG. 8 shows a schematic cross section through a further measuring cavity bar 400 which is similar or identical to the measuring cavity bar 300 except for the differences described below. In particular, in the measuring cavity bar 400, the additional heating element 310 is formed as a doped region in the semiconductor substrate 116, while the two conductor elements 106a, 106b are formed in a structured metal layer. In other words, the configuration of the additional heating element 310 and the conductor elements 106a, 106b is reversed compared to the measuring cavity bar 300.

According to one example, in the measuring cavity bar 400, the additional heating element 310 can also be omitted, e.g., the measuring cavity bar 400 has the two conductor elements 106a, 106b which are formed in a metallization on the semiconductor material of the measuring cavity bar 400.

In a gas sensor chip 100, in which the two conductor elements of the measuring and reference cavity bars are equivalent, as was shown with reference to FIGS. 6-8, the conductor elements can be interchanged without the measurement result being distorted. This can be used e.g., for diagnosing the gas sensor chip 100, for example to identify a defective conductor element. For this purpose, the circuits 122, 124 can be configured such that the position of a conductor element in the respective circuit of circuits 122, 124 or the association of a conductor element with one of the circuits 122, 124 can be changed.

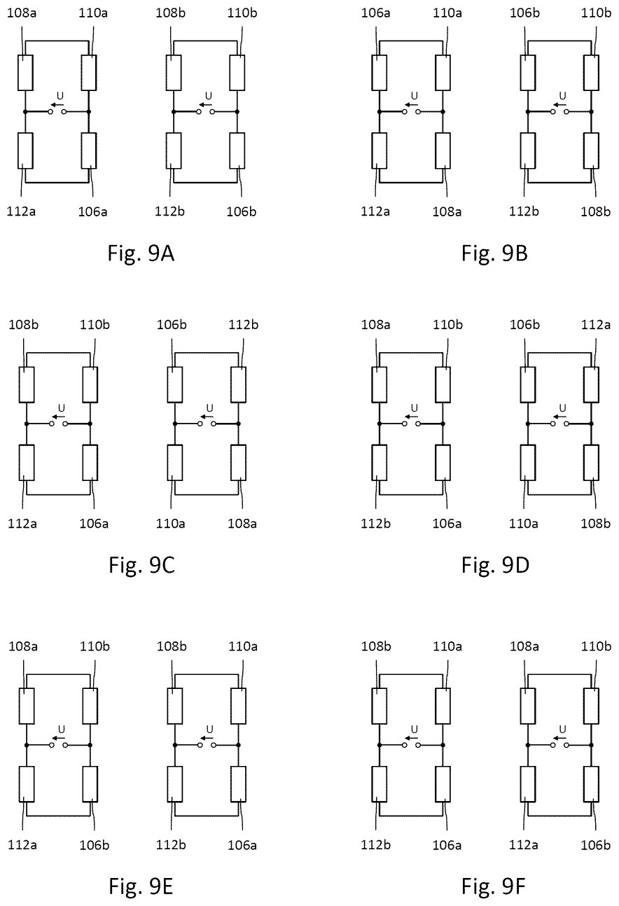

FIGS. 9A to 9F show various example Wheatstone bridge circuit arrangements in which the individual conductor elements are arranged at different positions in two circuits. For the sake of simplicity, the voltage sources of the respectively illustrated circuits were omitted in FIGS. 9A to 9F (cf. FIG. 3).

In the circuit arrangements shown in FIGS. 9A and 9B, all first conductor elements 106a, 108a, 110a, 112a are part of a first circuit and all second conductor elements 106b, 108b, 110b, 112b are part of a second circuit. In FIGS. 9C to 9F by contrast, the first conductor elements 106a, 108a, 110a, 112a and the second conductor elements 106b, 108b, 110b, 112b are apportioned to both of the circuits.

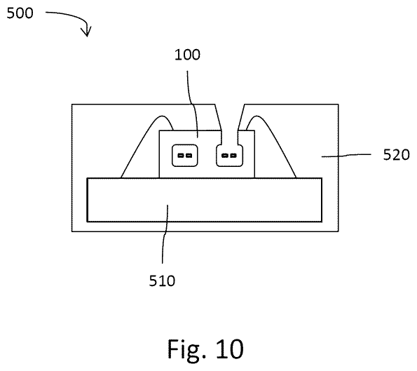

FIG. 10 shows a gas sensor 500 which has the gas sensor chip 100 and a control chip 510. The control chip 510 is configured to control the gas sensor chip 100. This means that the control chip 510 is for example configured to operate the gas sensor chip 100 in the different operating modes described here. The control chip 510 can be e.g., an ASIC (Application Specific Integrated Circuit). The gas sensor 500 may further have an encapsulation 520 which encapsulates the gas sensor chip 100 and the control chip 510 at least partially and is configured to protect the gas sensor chip 100 and the control chip 510 from environmental influences. The encapsulation 520 may comprise e.g., a moulded body or a plastic shell or consist of same.

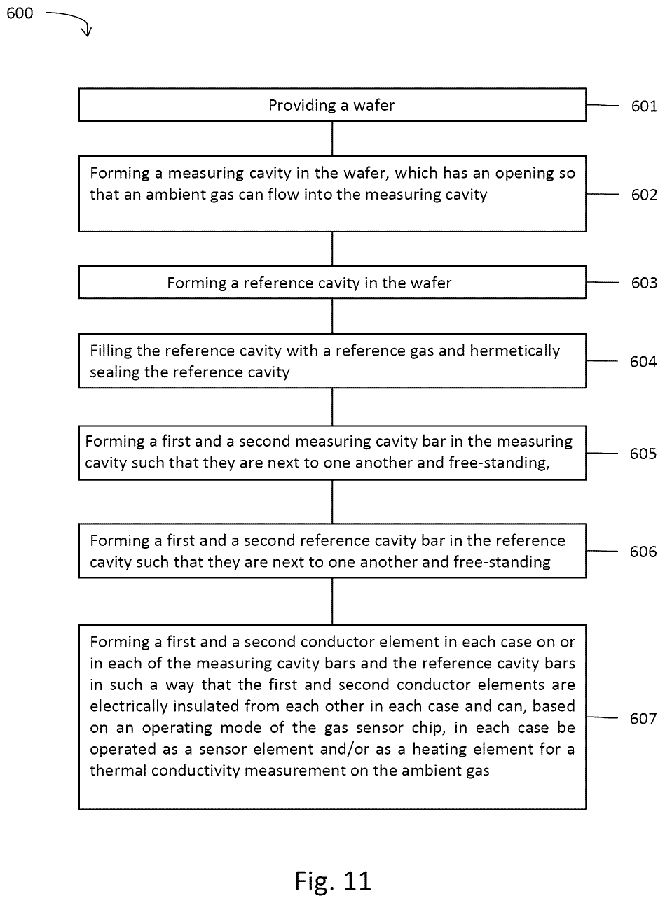

FIG. 11 is a flowchart of an example method 600 for producing a gas sensor chip for thermal conductivity measurements. The method 600 can be used e.g., to produce the gas sensor chip 100.

The method 600 comprises, at 601, a process of providing a wafer; at 602, a process of forming a measuring cavity in the wafer, which has an opening so that an ambient gas can flow into the measuring cavity; at 603, a process of forming a reference cavity in the wafer; at 604, a process of filling the reference cavity with a reference gas and hermetically sealing the reference cavity; at 605, a process of forming a first and a second measuring cavity bar in the measuring cavity such that they are next to one another and free-standing; at 606, a process of forming a first and a second reference cavity bar in the reference cavity such that they are next to one another and free-standing; and at 607, a process of forming a first and a second conductor element in each case on or in each of the measuring cavity bars and the reference cavity bars in such a way that the first and second conductor elements are electrically insulated from each other in each case and can, based on an operating mode of the gas sensor chip, in each case be operated as a sensor element and/or as a heating element for a thermal conductivity measurement on the ambient gas.

According to one example, the method 600 comprises a process of arranging an upper cover wafer over a wafer and a process of arranging a lower cover wafer under the wafer. In this case, an upper part of the measuring cavity and the reference cavity are formed in the upper cover wafer and a lower part of the measuring cavity and the reference cavity are formed in the lower cover wafer, wherein the opening of the measuring cavity is formed in the lower cover wafer.

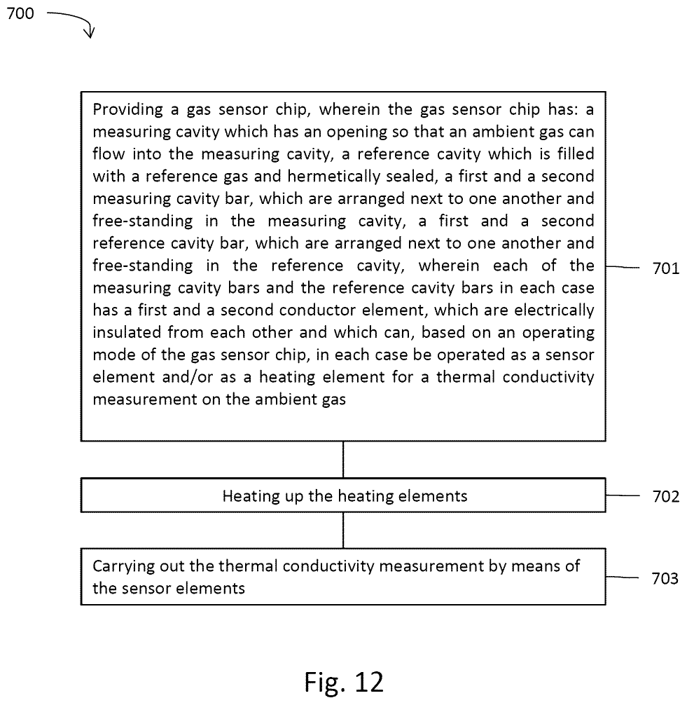

FIG. 12 is a flowchart of an example method 700 for operating a gas sensor chip for thermal conductivity measurements. The method 700 can e.g., be used to operate the gas sensor chip 100 in the manner described herein.

The method 700 comprises, at 701, a process of providing a gas sensor chip, wherein the gas sensor chip has: a measuring cavity which has an opening so that an ambient gas can flow into the measuring cavity, a reference cavity which is filled with a reference gas and hermetically sealed, a first and a second measuring cavity bar, which are arranged next to one another and free-standing in the measuring cavity, a first and a second reference cavity bar, which are arranged next to one another and free-standing in the reference cavity, wherein each of the measuring cavity bars and the reference cavity bars in each case has a first and a second conductor element, which are electrically insulated from each other and which can, based on an operating mode of the gas sensor chip, in each case be operated as a sensor element and/or as a heating element for a thermal conductivity measurement on the ambient gas; at 702, the method 700 comprises a process of heating up of the heating elements; and at 703, a process of carrying out the thermal conductivity measurement using the sensor elements.

### ASPECTS

The gas sensor chip, the method for producing a gas sensor chip and the method for operating a gas sensor chip are explained in more detail below based on explicit aspects.

Aspect 1 is a gas sensor chip for carrying out a thermal conductivity measurement, having: a measuring cavity which has an opening so that an ambient gas can flow into the measuring cavity, a reference cavity which is filled with a reference gas and hermetically sealed, a first and a second measuring cavity bar, which are arranged next to one another and free-standing in the measuring cavity, a first and a second reference cavity bar, which are arranged next to one another and free-standing in the reference cavity, wherein each of the measuring cavity bars and the reference cavity bars in each case has a first and a second conductor element, which are electrically insulated from each other and which can, based on an operating mode of the gas sensor chip, in each case be operated as a sensor element and/or as a heating element for a thermal conductivity measurement on the ambient gas.

Aspect 2 is the gas sensor chip according to aspect 1, wherein the measuring cavity bars and the reference cavity bars in each case comprise a semiconductor material or consist of same, wherein side walls of the measuring cavity and side walls of the reference cavity at least partially consist of the semiconductor material, and wherein the measuring cavity bars are formed monolithically with the semiconductor material of the side walls of the measuring cavity and the reference cavity bars are formed monolithically with the semiconductor material of the side walls of the reference cavity.

Aspect 3 is the gas sensor chip according to aspect 1 or 2, wherein the first conductor element of the measuring cavity bars and reference cavity bars in each case comprises a doped semiconductor material or consists of same, and wherein the second conductor element of the measuring cavity bars and reference cavity bars in each case comprises a metal or a metal alloy or consists of same.

Aspect 4 is the gas sensor chip according to aspect 1 or 2, wherein the first conductor element and the second conductor element of the measuring cavity bars and reference cavity bars in each case comprise a doped semiconductor material or consist of same.

Aspect 5 is the gas sensor chip according to aspect 4, further having: first additional heating elements which are arranged on the measuring cavity bars, second additional heating elements which are arranged on the reference cavity bars, wherein the first and second additional heating elements comprise a metal or a metal alloy or consist of same.

Aspect 6 is the gas sensor chip according to aspect 1 or 2, wherein the first conductor element and the second conductor element of the measuring cavity bars and reference cavity bars in each case comprise a metal or a metal alloy or consist of same.

Aspect 7 is the gas sensor chip according to aspect 6, further having: first additional heating elements which are formed in the measuring cavity bars, second additional heating elements which are formed in the reference cavity bars, wherein the first and second additional heating elements comprise a doped semiconductor material or consist of same.

Aspect 8 is the gas sensor chip according to one of the preceding aspects, wherein the first conductor elements of the measuring cavity bars and the reference cavity bars are part of a first circuit and the second conductor elements of the measuring cavity bars and the reference cavity bars are part of a second circuit, wherein, based on an operating mode of the gas sensor chip, the first circuit and the second circuit can be switched off or can be operated as a heating circuit or as a measuring circuit independently of each other in each case, wherein in the case of operation as a heating circuit, the conductor elements of the respective circuit are parallel-connected, and in the case of operation as a measuring circuit, the conductor elements of the respective circuit are connected in a Wheatstone bridge circuit arrangement.

Aspect 9 is the gas sensor chip according to aspect 8, wherein the gas sensor chip is configured to be operated in different sequences of operating modes in each case, based on the temperature at which the thermal conductivity measurement should be carried out.

Aspect 10 is the gas sensor chip according to aspect 9, wherein the gas sensor chip is at least configured to carry out the thermal conductivity measurement at a comparatively low temperature, a comparatively moderate temperature, and a comparatively high temperature.

Aspect 11 is the gas sensor chip according to any one of aspects 8 to 10, wherein in one of the operating modes, both circuits are operated as a measuring circuit simultaneously to provide redundancy of the thermal conductivity measurement.

Aspect 12 is the gas sensor chip according to any one of aspects 8 to 11, wherein the gas sensor chip is configured such that, based on an operating mode of the gas sensor chip, the interconnection of the first and second conductor elements is changed in such a way that one or more of the first conductor elements of the first circuit supplant(s) one or more of the second conductor elements of the second circuit and vice versa.

Aspect 13 is a gas sensor for carrying out a thermal conductivity measurement, having: the gas sensor chip according to any one of aspects 1 to 12, a control chip which is configured to operate the gas sensor chip in different operating modes, and an encapsulation which encapsulates the gas sensor chip and the control chip.

Aspect 14 is a method for producing a gas sensor chip for thermal conductivity measurements, wherein the method comprises: providing a wafer, forming a measuring cavity in the wafer, which has an opening so that an ambient gas can flow into the measuring cavity, forming a reference cavity in the wafer, filling the reference cavity with a reference gas and hermetically sealing the reference cavity, forming a first and a second measuring cavity bar in the measuring cavity such that they are next to one another and free-standing, forming a first and a second reference cavity bar in the reference cavity such that they are next to one another and free-standing, forming a first and a second conductor element in each case on or in each of the measuring cavity bars and the reference cavity bars in such a way that the first and second conductor elements are electrically insulated from each other in each case and can, based on an operating mode of the gas sensor chip, in each case be operated as a sensor element and/or as a heating element for a thermal conductivity measurement on the ambient gas.

Aspect 15 is the method according to aspect 14, wherein the formation of the first conductor elements of the measuring cavity bars and reference cavity bars comprises doping of a semiconductor material, and wherein the formation of the second conductor elements of the measuring cavity bars and reference cavity bars comprises deposition of a metal or a metal alloy onto the measuring cavity bars and reference cavity bars.

Aspect 16 is the method according to either of aspects 14 and 15, further comprising: forming a first electrical circuit arrangement in the wafer, which circuit arrangement has the first conductor elements, and forming a second electrical circuit arrangement in the wafer, which circuit arrangement has the second conductor elements, wherein the first and the second electrical circuit arrangement are insulated from each other.

Aspect 17 is a method for operating a gas sensor chip for thermal conductivity measurements, the method comprising: providing a gas sensor chip having: a measuring cavity which has an opening so that an ambient gas can flow into the measuring cavity, a reference cavity which is filled with a reference gas and hermetically sealed, a first and a second measuring cavity bar, which are arranged next to one another and free-standing in the measuring cavity, a first and a second reference cavity bar, which are arranged next to one another and free-standing in the reference cavity, wherein each of the measuring cavity bars and the reference cavity bars in each case has a first and a second conductor element, which are electrically insulated from each other and which can, based on an operating mode of the gas sensor chip, in each case be operated as a sensor element and/or as a heating element for a thermal conductivity measurement on the ambient gas; heating up the heating elements; and carrying out the thermal conductivity measurement using the sensor elements.

Aspect 18 is the method according to aspect 17, wherein the first conductor elements of the measuring cavity bars and the reference cavity bars are part of a first circuit and the second conductor elements of the measuring cavity bars and the reference cavity bars are part of a second circuit, wherein, based on an operating mode of the gas sensor chip, the first circuit and the second circuit are switched off or are operated as a heating circuit or as a measuring circuit independently of each other in each case, wherein in the case of operation as a heating circuit, the conductor elements of the respective circuit are parallel-connected, and in the case of operation as a measuring circuit, the conductor elements of the respective circuit are connected in a Wheatstone bridge circuit arrangement.

Aspect 19 is the method according to aspect 18, further comprising: operating the gas sensor chip in an operating mode in which both circuits are operated as a heating circuit for heating up the ambient gas and the reference gas, and switching to a further operating mode in which both circuits are operated as a measuring circuit for carrying out the thermal conductivity measurement.

Aspect 20 is the method according to aspect 18, further comprising: operating the gas sensor chip in an operating mode in which both circuits are operated as a measuring circuit for carrying out a first thermal conductivity measurement at a comparatively low temperature, switching to a further operating mode in which both circuits are operated as a heating circuit for heating up the measuring cavity bars and the reference cavity bars, and switching to the operating mode in which both circuits are operated as a measuring circuit for carrying out a second thermal conductivity measurement at a comparatively high temperature.

Aspect 21 is the method according to aspect 18, further comprising: carrying out temperature spectroscopy on the ambient gas, wherein the temperature spectroscopy comprises: operating the gas sensor chip in a sequence of alternating operating modes and thus setting alternating temperatures of the reference gas and the ambient gas, and carrying out the thermal conductivity measurement at the alternating temperatures.

Aspect 22 is the method according to any one of aspects 18 to 21, further comprising: changing interconnection of the first and second conductor elements in such a way that one or more of the first conductor elements of the first circuit supplant(s) one or more of the second conductor elements of the second circuit and vice versa, and carrying out the thermal conductivity measurement with the changed interconnection.

Aspect 23 is the method according to aspect 22, wherein the measuring cavity bars and the reference cavity bars in each case have additional heating elements, and heating the reference gas and the ambient gas using the additional heating elements in the changed interconnection.

Aspect 24 is a device having means for carrying out the method according to any one of aspects 14 to 23.

The following provides an overview of some additional aspects of the present disclosure:

Additional aspect 1: A gas sensor chip for carrying out a thermal conductivity measurement, comprising: a measuring cavity which has an opening so that an ambient gas can flow into the measuring cavity; a reference cavity which is filled with a reference gas and hermetically sealed; a first measuring cavity bar and a second measuring cavity bar, which are arranged next to one another and free-standing in the measuring cavity; and a first reference cavity bar and a second reference cavity bar, which are arranged next to one another and free-standing in the reference cavity, wherein each of the first measuring cavity bar, the second measuring cavity bar, the first reference cavity bar, and the second reference cavity bar and the reference cavity bars have a first conductor element and a second conductor element, which are electrically insulated from each other and which are, based on an operating mode of a plurality of operating modes of the gas sensor chip, in each case, configured to be operated as at least one of a sensor element or a heating element for a thermal conductivity measurement on the ambient gas.

Additional aspect 2: The gas sensor chip as recited in Additional aspect 1, wherein first measuring cavity bar, the second measuring cavity bar, the first reference cavity bar, and the second reference cavity bar, in each case, comprise a semiconductor material, wherein side walls of the measuring cavity and side walls of the reference cavity at least partially consist of the semiconductor material, and wherein the first measuring cavity bar and the second measuring cavity bar are formed monolithically with the semiconductor material of the side walls of the measuring cavity, and the first reference cavity bar and the second reference cavity bar are formed monolithically with the semiconductor material of the side walls of the reference cavity.

Additional aspect 3: The gas sensor chip as claimed in any of Additional aspects 1-2, wherein the first conductor element of the first measuring cavity bar, the second measuring cavity bar, the first reference cavity bar, and the second reference cavity bar, in each case, comprises a doped semiconductor material, and wherein the second conductor element of the first measuring cavity bar, the second measuring cavity bar, the first reference cavity bar, and the second reference cavity bar, in each case, comprises a metal or a metal alloy.

Additional aspect 4: The gas sensor chip as claimed in any of Additional aspects 1-3, wherein the first conductor element and the second conductor element of the first measuring cavity bar, the second measuring cavity bar, the first reference cavity bar, and the second reference cavity bar, in each case, comprise a doped semiconductor material.

Additional aspect 5: The gas sensor chip as recited in Additional aspect 4, further comprising: first additional heating elements arranged on the first measuring cavity bar and the second measuring cavity bar; and second additional heating elements arranged on the first reference cavity bar and the second reference cavity bar, wherein the first additional heating elements and the second additional heating elements comprise a metal or a metal alloy.

Additional aspect 6: The gas sensor chip as claimed in any of Additional aspects 1-5, wherein the first conductor element and the second conductor element of the first measuring cavity bar, the second measuring cavity bar, the first reference cavity bar, and the second reference cavity bar, in each case, comprise a metal or a metal alloy or consist of same.

Additional aspect 7: The gas sensor chip as recited in Additional aspect 6, further comprising: first additional heating elements formed in the first measuring cavity bar and the second measuring cavity bar; and second additional heating elements formed in the first reference cavity bar and the second reference cavity bar, wherein the first additional heating elements and the second additional heating elements comprise a doped semiconductor material.

Additional aspect 8: The gas sensor chip as claimed in any of Additional aspects 1-7, wherein the first conductor elements of the first measuring cavity bar, the second measuring cavity bar, the first reference cavity bar, and the second reference cavity bar are part of a first circuit and the second conductor elements of the first measuring cavity bar, the second measuring cavity bar, the first reference cavity bar, and the second reference cavity bar are part of a second circuit, wherein, based on the operating mode of the plurality of operating modes of the gas sensor chip, the first circuit and the second circuit are configured to be switched off, operated as a heating circuit, or operated as a measuring circuit independently of each other in each case, and wherein in the case of operating as a heating circuit, the first conductor elements of the first circuit are parallel-connected, and the second conductor elements of the second circuit are parallel-connected, and in the case of operating as a measuring circuit, the first conductor elements of the first circuit are connected in a first Wheatstone bridge circuit arrangement, and the second conductor elements of the second circuit are connected in a second Wheatstone bridge circuit arrangement.

Additional aspect 9: The gas sensor chip as recited in Additional aspect 8, wherein the gas sensor chip is configured to be operated in different sequences of operating modes, in each case, based on a temperature at which the thermal conductivity measurement is carried out.

Additional aspect 10: The gas sensor chip as recited in Additional aspect 9, wherein the gas sensor chip is configured to carry out the thermal conductivity measurement at a comparatively low temperature, a comparatively moderate temperature, or a comparatively high temperature.

Additional aspect 11: The gas sensor chip as recited in Additional aspect 8, wherein in one of the plurality of operating modes, both the first circuit and the second circuit are operated as measuring circuits simultaneously to provide redundancy of the thermal conductivity measurement.

Additional aspect 12: The gas sensor chip as recited in Additional aspect 8, wherein the gas sensor chip is configured such that, based on an operating mode of the gas sensor chip, an interconnection of the first conductor elements and the second conductor elements is changed in such a way that one or more of the first conductor elements of the first circuit supplant one or more of the second conductor elements of the second circuit, or the interconnection of the first conductor elements and the second conductor elements is changed in such a way that one or more of the second conductor elements of the second circuit supplant one or more of the first conductor elements of the first circuit.

Additional aspect 13: A gas sensor for carrying out a thermal conductivity measurement, comprising: a gas sensor chip comprising: a measuring cavity which has an opening so that an ambient gas can flow into the measuring cavity; a reference cavity which is filled with a reference gas and hermetically sealed; a first measuring cavity bar and a second measuring cavity bar, which are arranged next to one another and free-standing in the measuring cavity; and a first reference cavity bar and a second reference cavity bar, which are arranged next to one another and free-standing in the reference cavity; wherein each of the first measuring cavity bar, the second measuring cavity bar, the first reference cavity bar, and the second reference cavity bar and the reference cavity bars have a first conductor element and a second conductor element, which are electrically insulated from each other and which are, based on an operating mode of the gas sensor chip, in each case, configured to be operated as at least one of a sensor element or a heating element for a thermal conductivity measurement on the ambient gas; a control chip configured to operate the gas sensor chip in different operating modes; and an encapsulation which encapsulates the gas sensor chip and the control chip.

Additional aspect 14: A method for producing a gas sensor chip for thermal conductivity measurements, wherein the method comprises: providing a wafer; forming a measuring cavity in the wafer, which has an opening so that an ambient gas can flow into the measuring cavity; forming a reference cavity in the wafer; filling the reference cavity with a reference gas and hermetically sealing the reference cavity; forming a first measuring cavity bar and a second measuring cavity bar in the measuring cavity such that the first measuring cavity bar and the second measuring cavity bar are next to one another and free-standing; forming a first reference cavity bar and a second reference cavity bar in the reference cavity such that the first reference cavity bar and the second reference cavity bar are next to one another and free-standing; and forming a first conductor element and a second conductor element, in each case, on or in each of the first measuring cavity bar, the second measuring cavity bar, the first reference cavity bar, and the second reference cavity bar, in such a way that the first conductor element and the second conductor element, in each case, are electrically insulated from each other and, based on an operating mode of a plurality of operating modes of the gas sensor chip, in each case are operable as at least one of a sensor element or as a heating element for a thermal conductivity measurement on the ambient gas.

Additional aspect 15: The method as recited in Additional aspect 14, wherein forming each first conductor element of the first measuring cavity bar, the second measuring cavity bar, the first reference cavity bar, and the second reference cavity bar comprises doping of a semiconductor material, and wherein forming each second conductor element of the first measuring cavity bar, the second measuring cavity bar, the first reference cavity bar, and the second reference cavity bar comprises deposition of a metal or a metal alloy onto the measuring cavity bars and reference cavity bars.

Additional aspect 16: The method as claimed in any of Additional aspects 14-15, further comprising: forming a first electrical circuit arrangement in the wafer, the first electrical circuit arrangement including the first conductor elements; and forming a second electrical circuit arrangement in the wafer, w the second electrical circuit arrangement including the second conductor elements, wherein the first electrical circuit arrangement and the second electrical circuit arrangement are insulated from each other.

Additional aspect 17: A method for operating a gas sensor chip for thermal conductivity measurements, the method comprising: providing a gas sensor chip comprising: a measuring cavity which has an opening so that an ambient gas can flow into the measuring cavity, a reference cavity which is filled with a reference gas and hermetically sealed, a first measuring cavity bar and a second measuring cavity bar, which are arranged next to one another and free-standing in the measuring cavity, a first reference cavity bar and a second reference cavity bar, which are arranged next to one another and free-standing in the reference cavity, wherein each of the first measuring cavity bar, the second measuring cavity bar, the first reference cavity bar, and the second reference cavity bar, in each case, have a first conductor element and a second conductor element, which are electrically insulated from each other and which are, based on an operating mode of a plurality of operating modes of the gas sensor chip, in each case, operable as at least one of a sensor element or a heating element for a thermal conductivity measurement on the ambient gas; heating up one or more first conductor elements and one or more second conductor elements configured as heating elements; and carrying out the thermal conductivity measurement using one or more first conductor elements and one or more second conductor elements configured as sensor elements.

Additional aspect 18: The method as recited in Additional aspect 17, wherein each first conductor element of the first measuring cavity bar, the second measuring cavity bar, the first reference cavity bar, and the second reference cavity bar is part of a first circuit and each second conductor element of the first measuring cavity bar, the second measuring cavity bar, the first reference cavity bar, and the second reference cavity bar is part of a second circuit, wherein, based on the operating mode of the gas sensor chip, the first circuit and the second circuit are switched off, operated as a heating circuit, or operated as a measuring circuit independently of each other, in each case, and wherein in the case of operating as a heating circuit, the first conductor elements of the first circuit are parallel-connected, and the second conductor elements of the second circuit are parallel-connected, and in the case of operating as a measuring circuit, the first conductor elements of the first circuit are connected in a first Wheatstone bridge circuit arrangement, and the second conductor elements of the second circuit are connected in a second Wheatstone bridge circuit arrangement.

Additional aspect 19: The method as recited in Additional aspect 18, further comprising: operating the gas sensor chip in an operating mode in which both the first circuit and the second circuit are operated as a heating circuit for heating up the ambient gas and the reference gas; and switching to a further operating mode in which both the first circuit and the second circuit are operated as a measuring circuit for carrying out a thermal conductivity measurement.

Additional aspect 20: The method as recited in Additional aspect 18, further comprising: operating the gas sensor chip in a first operating mode in which both the first circuit and the second circuit are operated as a measuring circuit for carrying out a first thermal conductivity measurement at a comparatively low temperature; switching to a second operating mode in which both the first circuit and the second circuit are operated as a heating circuit for heating up the first measuring cavity bar, the second measuring cavity bar, the first reference cavity bar, and the second reference cavity bar; and switching to a third operating mode in which both the first circuit and the second circuit are operated as a measuring circuit for carrying out a second thermal conductivity measurement at a comparatively high temperature.

Additional aspect 21: The method as recited in Additional aspect 18, further comprising: carrying out temperature spectroscopy on the ambient gas, wherein carrying out the temperature spectroscopy comprises: operating the gas sensor chip in a sequence of alternating operating modes and thus setting alternating temperatures of the reference gas and the ambient gas; and carrying out a thermal conductivity measurement at the alternating temperatures.

Additional aspect 22: The method as recited in Additional aspect 18, further comprising: changing an interconnection of the first conductor elements and the second conductor elements to form a changed interconnection in such a way that one or more of the first conductor elements of the first circuit supplant one or more of the second conductor elements of the second circuit, or changing the interconnection of the first conductor elements and the second conductor elements is changed in such a way that one or more of the second conductor elements of the second circuit supplant one or more of the first conductor elements of the first circuit; and carrying out a thermal conductivity measurement with the changed interconnection.

Additional aspect 23: The method as recited in Additional aspect 22, wherein the first measuring cavity bar, the second measuring cavity bar, the first reference cavity bar, and the second reference cavity bar, in each case, have additional heating elements, and the method further comprises: heating the reference gas and the ambient gas using the additional heating elements in the changed interconnection.

Additional aspect 24: A system configured to perform one or more operations recited in one or more of Additional aspects 1-23.

Additional aspect 25: An apparatus comprising means for performing one or more operations recited in one or more of Additional aspects 1-23.

It should be pointed out that the description and the drawings only illustrate the principles of the proposed methods and devices. A person skilled in the art will be capable of implementing different arrangements which, although they are not expressly described or shown here, embody the principles of the implementation and are contained within the scope thereof. In addition, all aspects and implementations outlined in the present document are intended fundamentally and expressly for explanatory purposes only, in order to help the reader understand the principles of the proposed methods and devices. In addition, all statements in this document which describe principles, aspects and implementations of the implementation and specific aspects thereof are also intended to comprise their equivalents.

1. A gas sensor chip for carrying out a thermal conductivity measurement, comprising:
a measuring cavity which has an opening so that an ambient gas can flow into the measuring cavity;
a reference cavity which is filled with a reference gas and hermetically sealed;
a first measuring cavity bar and a second measuring cavity bar, which are arranged next to one another and free-standing in the measuring cavity; and
a first reference cavity bar and a second reference cavity bar, which are arranged next to one another and free-standing in the reference cavity,
wherein each of the first measuring cavity bar, the second measuring cavity bar, the first reference cavity bar, and the second reference cavity bar and the reference cavity bars have a first conductor element and a second conductor element, which are electrically insulated from each other and which are, based on an operating mode of a plurality of operating modes of the gas sensor chip, in each case, configured to be operated as at least one of a sensor element or a heating element for a thermal conductivity measurement on the ambient gas.

2. The gas sensor chip as claimed in claim 1,
wherein first measuring cavity bar, the second measuring cavity bar, the first reference cavity bar, and the second reference cavity bar, in each case, comprise a semiconductor material,
wherein side walls of the measuring cavity and side walls of the reference cavity at least partially consist of the semiconductor material, and
wherein the first measuring cavity bar and the second measuring cavity bar are formed monolithically with the semiconductor material of the side walls of the measuring cavity, and the first reference cavity bar and the second reference cavity bar are formed monolithically with the semiconductor material of the side walls of the reference cavity.

3. The gas sensor chip as claimed in claim 1,
wherein the first conductor element of the first measuring cavity bar, the second measuring cavity bar, the first reference cavity bar, and the second reference cavity bar, in each case, comprises a doped semiconductor material, and
wherein the second conductor element of the first measuring cavity bar, the second measuring cavity bar, the first reference cavity bar, and the second reference cavity bar, in each case, comprises a metal or a metal alloy.

4. The gas sensor chip as claimed in claim 1, wherein the first conductor element and the second conductor element of the first measuring cavity bar, the second measuring cavity bar, the first reference cavity bar, and the second reference cavity bar, in each case, comprise a doped semiconductor material.

5. The gas sensor chip as claimed in claim 4, further comprising:
first additional heating elements arranged on the first measuring cavity bar and the second measuring cavity bar; and
second additional heating elements arranged on the first reference cavity bar and the second reference cavity bar,
wherein the first additional heating elements and the second additional heating elements comprise a metal or a metal alloy.

6. The gas sensor chip as claimed in claim 1, wherein the first conductor element and the second conductor element of the first measuring cavity bar, the second measuring cavity bar, the first reference cavity bar, and the second reference cavity bar, in each case, comprise a metal or a metal alloy or consist of same.

7. The gas sensor chip as claimed in claim 6, further comprising:
first additional heating elements formed in the first measuring cavity bar and the second measuring cavity bar; and
second additional heating elements formed in the first reference cavity bar and the second reference cavity bar,
wherein the first additional heating elements and the second additional heating elements comprise a doped semiconductor material.

8. The gas sensor chip as claimed in claim 1,
wherein the first conductor elements of the first measuring cavity bar, the second measuring cavity bar, the first reference cavity bar, and the second reference cavity bar are part of a first circuit and the second conductor elements of the first measuring cavity bar, the second measuring cavity bar, the first reference cavity bar, and the second reference cavity bar are part of a second circuit,
wherein, based on the operating mode of the plurality of operating modes of the gas sensor chip, the first circuit and the second circuit are configured to be switched off, operated as a heating circuit, or operated as a measuring circuit independently of each other in each case, and
wherein in the case of operating as a heating circuit, the first conductor elements of the first circuit are parallel-connected, and the second conductor elements of the second circuit are parallel-connected,
and in the case of operating as a measuring circuit, the first conductor elements of the first circuit are connected in a first Wheatstone bridge circuit arrangement, and the second conductor elements of the second circuit are connected in a second Wheatstone bridge circuit arrangement.

9. The gas sensor chip as claimed in claim 8, wherein the gas sensor chip is configured to be operated in different sequences of operating modes, in each case, based on a temperature at which the thermal conductivity measurement is carried out.

10. The gas sensor chip as claimed in claim 9, wherein the gas sensor chip is configured to carry out the thermal conductivity measurement at a comparatively low temperature, a comparatively moderate temperature, or a comparatively high temperature.

11. The gas sensor chip as claimed in claim 8, wherein in one of the plurality of operating modes, both the first circuit and the second circuit are operated as measuring circuits simultaneously to provide redundancy of the thermal conductivity measurement.

12. The gas sensor chip as claimed in claim 8, wherein the gas sensor chip is configured such that, based on an operating mode of the gas sensor chip, an interconnection of the first conductor elements and the second conductor elements is changed in such a way that one or more of the first conductor elements of the first circuit supplant one or more of the second conductor elements of the second circuit, or the interconnection of the first conductor elements and the second conductor elements is changed in such a way that one or more of the second conductor elements of the second circuit supplant one or more of the first conductor elements of the first circuit.

13. A gas sensor for carrying out a thermal conductivity measurement, comprising:
a gas sensor chip comprising:
a measuring cavity which has an opening so that an ambient gas can flow into the measuring cavity;
a reference cavity which is filled with a reference gas and hermetically sealed;
a first measuring cavity bar and a second measuring cavity bar, which are arranged next to one another and free-standing in the measuring cavity; and
a first reference cavity bar and a second reference cavity bar, which are arranged next to one another and free-standing in the reference cavity;
wherein each of the first measuring cavity bar, the second measuring cavity bar, the first reference cavity bar, and the second reference cavity bar and the reference cavity bars have a first conductor element and a second conductor element, which are electrically insulated from each other and which are, based on an operating mode of the gas sensor chip, in each case, configured to be operated as at least one of a sensor element or a heating element for a thermal conductivity measurement on the ambient gas;
a control chip configured to operate the gas sensor chip in different operating modes; and
an encapsulation which encapsulates the gas sensor chip and the control chip.

14. A method for producing a gas sensor chip for thermal conductivity measurements, wherein the method comprises:
providing a wafer;
forming a measuring cavity in the wafer, which has an opening so that an ambient gas can flow into the measuring cavity;
forming a reference cavity in the wafer;
filling the reference cavity with a reference gas and hermetically sealing the reference cavity;
forming a first measuring cavity bar and a second measuring cavity bar in the measuring cavity such that the first measuring cavity bar and the second measuring cavity bar are next to one another and free-standing;
forming a first reference cavity bar and a second reference cavity bar in the reference cavity such that the first reference cavity bar and the second reference cavity bar are next to one another and free-standing; and
forming a first conductor element and a second conductor element, in each case, on or in each of the first measuring cavity bar, the second measuring cavity bar, the first reference cavity bar, and the second reference cavity bar, in such a way that the first conductor element and the second conductor element, in each case, are electrically insulated from each other and based on an operating mode of a plurality of operating modes of the gas sensor chip, in each case are operable as at least one of a sensor element or as a heating element for a thermal conductivity measurement on the ambient gas.

15. The method as claimed in claim 14,
wherein forming each first conductor element of the first measuring cavity bar, the second measuring cavity bar, the first reference cavity bar, and the second reference cavity bar comprises doping of a semiconductor material, and
wherein forming each second conductor element of the first measuring cavity bar, the second measuring cavity bar, the first reference cavity bar, and the second reference cavity bar comprises deposition of a metal or a metal alloy onto the measuring cavity bars and reference cavity bars.

16. The method as claimed in claim 14, further comprising:
forming a first electrical circuit arrangement in the wafer, the first electrical circuit arrangement including the first conductor elements; and
forming a second electrical circuit arrangement in the wafer, the second electrical circuit arrangement including the second conductor elements,
wherein the first electrical circuit arrangement and the second electrical circuit arrangement are insulated from each other.

17. A method for operating a gas sensor chip for thermal conductivity measurements, the method comprising:
providing a gas sensor chip comprising:
a measuring cavity which has an opening so that an ambient gas can flow into the measuring cavity,
a reference cavity which is filled with a reference gas and hermetically sealed,
a first measuring cavity bar and a second measuring cavity bar, which are arranged next to one another and free-standing in the measuring cavity,
a first reference cavity bar and a second reference cavity bar, which are arranged next to one another and free-standing in the reference cavity,
wherein each of the first measuring cavity bar, the second measuring cavity bar, the first reference cavity bar, and the second reference cavity bar, in each case, have a first conductor element and a second conductor element, which are electrically insulated from each other and which are, based on an operating mode of a plurality of operating modes of the gas sensor chip, in each case, operable as at least one of a sensor element or a heating element for a thermal conductivity measurement on the ambient gas;
heating up one or more first conductor elements and one or more second conductor elements configured as heating elements; and
carrying out the thermal conductivity measurement using one or more first conductor elements and one or more second conductor elements configured as sensor elements.

18. The method as claimed in claim 17,
wherein each first conductor element of the first measuring cavity bar, the second measuring cavity bar, the first reference cavity bar, and the second reference cavity bar is part of a first circuit and each second conductor element of the first measuring cavity bar, the second measuring cavity bar, the first reference cavity bar, and the second reference cavity bar is part of a second circuit,
wherein, based on the operating mode of the gas sensor chip, the first circuit and the second circuit are switched off, operated as a heating circuit, or operated as a measuring circuit independently of each other, in each case, and
wherein in the case of operating as a heating circuit, the first conductor elements of the first circuit are parallel-connected, and the second conductor elements of the second circuit are parallel-connected, and
in the case of operating as a measuring circuit, the first conductor elements of the first circuit are connected in a first Wheatstone bridge circuit arrangement, and the second conductor elements of the second circuit are connected in a second Wheatstone bridge circuit arrangement.

19. The method as claimed in claim 18, further comprising:
operating the gas sensor chip in an operating mode in which both the first circuit and the second circuit are operated as a heating circuit for heating up the ambient gas and the reference gas; and
switching to a further operating mode in which both the first circuit and the second circuit are operated as a measuring circuit for carrying out a thermal conductivity measurement.

20. The method as claimed in claim 18, further comprising:
operating the gas sensor chip in a first operating mode in which both the first circuit and the second circuit are operated as a measuring circuit for carrying out a first thermal conductivity measurement at a comparatively low temperature;
switching to a second operating mode in which both the first circuit and the second circuit are operated as a heating circuit for heating up the, first measuring cavity bar, the second measuring cavity bar, the first reference cavity bar, and the second reference cavity bar; and
switching to a third operating mode in which both the first circuit and the second circuit are operated as a measuring circuit for carrying out a second thermal conductivity measurement at a comparatively high temperature.

21. The method as claimed in claim 18, further comprising:
carrying out temperature spectroscopy on the ambient gas, wherein carrying out the temperature spectroscopy comprises:
operating the gas sensor chip in a sequence of alternating operating modes and thus setting alternating temperatures of the reference gas and the ambient gas; and
carrying out a thermal conductivity measurement at the alternating temperatures.

22. The method as claimed in claim 18, further comprising:
changing an interconnection of the first conductor elements and the second conductor elements to form a changed interconnection in such a way that one or more of the first conductor elements of the first circuit supplant one or more of the second conductor elements of the second circuit, or changing the interconnection of the first conductor elements and the second conductor elements is changed in such a way that one or more of the second conductor elements of the second circuit supplant one or more of the first conductor elements of the first circuit; and
carrying out a thermal conductivity measurement with the changed interconnection.

23. The method as claimed in claim 22,
wherein the first measuring cavity bar, the second measuring cavity bar, the first reference cavity bar, and the second reference cavity bar, in each case, have additional heating elements, and
the method further comprises:
heating the reference gas and the ambient gas using the additional heating elements in the changed interconnection.
